(19) United States
(12) Patent Application Publication
(10) Pub. No.: US 2004/0248825 A1
(43) Pub. Date: Dec. 9, 2004

(54) Use of neuraminidase inhibitors to prevent flu associated bacterial infections

(75) Inventor: Jonathan A. McCullers, Germantown, TN (US)

Correspondence Address: ALSTON AND BIRD LLP, ST. JUDE CHILDREN'S RESEARCH HOSPITAL, BANK OF AMERICA PLAZA, 101 SOUTH TRYON STREET, SUITE 4000, CHARLOTTE, NC, 28280-4000, US

(73) Assignee: St. Jude Children's Research Hospital, Memphis, TN

(21) Appl. No.: 10/809,127

(22) Filed: Mar. 25, 2004

Related U.S. Application Data
(63) Continuation of application No. PCT/US02/29417, filed on Sep. 17, 2002.
(60) Provisional application No. 60/325,615, filed on Sep. 27, 2001.

(51) Int. Cl. A61K 31/7056; A61K 31/551

(52) U.S. Cl. 514/43; 514/220

(57) ABSTRACT

The invention provides a novel use for neuraminidase inhibitors in chemoprophylactic and treatment methods for the prevention, attenuation and treatment of bacterial infections that may occur in association with, or as a sequelae of, viral influenza. The prophylactic methods of the invention are particularly suitable for the prevention of secondary bacterial infections, such as, but not limited to infections of the lower respiratory tract (e.g., pneumonia), middle ear infections (e.g., otitis media) and bacterial sinusitis. The treatment methods are suitable for use in protocols designed to attenuate or treat bacterial infections that occur concurrent with, or as a sequelae of the flu.

### CROSS-REFERENCE TO RELATED APPLICATIONS

This application is a U.S. continuation of PCT application No. PCT/US02/29417, filed Sep. 17, 2002, which was published in English on Apr. 3, 2003 as International Publication Number WO 03/02657 A2, and which claims priority to U.S. Provisional Application No. 60/325,615, filed Sep. 27, 2001, each of which is hereby incorporated in its entirety by reference herein.

### STATEMENT OF FEDERALLY SPONSORED RESEARCH

The research underlying this invention was supported in part with funds from Public Health research grants 1 K08 A149178-01, AI-08831 and AI-29680 from the National Institute of Allergy and Infectious Diseases and National Cancer Institute grant CA-21765 (Cancer Center CORE grant). The United States Government may have an interest in the subject matter of the invention.

### FIELD OF THE INVENTION

The present invention relates generally to clinical applications for neuraminidase inhibitors and to the treatment of bacterial infections associated with the flu.

### BACKGROUND OF THE INVENTION

Influenza, more commonly known as the flu, is an acute, viral infection of the respiratory tract that accounts for significant levels of morbidity and mortality. The Centers for Disease Control and Prevention (CDC) estimates that approximately 10% to 20% of the general population of the United States are afflicted with the flu annually resulting in approximately 20,000 influenza-associated deaths and more than 110,000 hospitalizations (Neuzil, et al., N. Eng. J. Med. (2000) 342:225-231). Uncomplicated influenza is characterized by the abrupt onset of constitutional (e.g., fever, myalgia, headache, severe malaise) and respiratory symptoms (e.g., nonproductive cough, sore throat and rhinitis). The flu is particularly dangerous for young children, elderly individuals, and for chronically ill patients. Direct costs associated with providing medical care for individuals afflicted with influenza have been estimated at between $1 billion and $3 billion annually.

Influenza viruses cause disease in all age groups. Children experience the highest incidence of influenza disease, two to ten times that seen in adults, and are a common source of its spread. However, rates of serious illness are highest among individuals who are 65 years of age or older, and people of any age who have medical conditions that place them at high risk for developing complications from the flu. Although healthy adults under the age of 65 are at a comparatively low risk for severe illness, the flu can result in substantial mortality, numerous health-provider visits and lost workdays. Estimates of annual indirect costs resulting from absenteeism from school and work and reduced productivity range from $10 to $15 billion.

Otitis media, bacterial sinusitis, and pneumonia are common complications of an infection with viral influenza. Otitis media and acute bacterial sinusitis are two of five conditions that account for most of the outpatient use of antibiotics in the United States. Data from the National Center for Health Statistics indicate that approximately 75% of all outpatient prescriptions for antibiotics are written for otitis media, sinusitis, bronchitis, pharyngitis and nonspecific upper respiratory tract infection.

The existence of a lethal synergism between influenza virus and bacteria such as S. pneumoniae has been appreciated since the early part of last century following the influenza pandemic of 1918-1919 (Abrahams, et al., Lancet (1919) 1-11; Hers et al.,(1958)Lancet, 2:1164-5; Hament et al.,(1999) FEMS Immunol Med Microbiol., 26(3-4):189-95; Heikkinen T., (2001) Vaccine, 19:S51-55; Simonsen L., (1999) Vaccine, 17:S3-10 and O'Brien et al.,(2000) Clin. Inf. Dis., 30:784-9), and continues to be a major medical problem that accounts for excess mortality during influenza epidemics worldwide. Generally speaking, the effects of a pathogenic synergism between the influenza virus and other bacterial pathogen can promote the development of severe bacterial infections in individuals afflicted with the flu. For example, a pathogenic synergism between the influenza virus and pneumococcus (i.e., Streptococcus pneumoniae) is known to cause a high incidence of lower respiratory tract infections during influenza epidemics. Simonsen, L., (1999) Vaccine 17: S3-S10; Klimov et al., (1999) Vaccine 17: S42-S46. The viral/bacterial synergism is evidenced by the fact that influenza and bacterial pneumonia taken together are the sixth leading cause of death in the world and the leading infectious cause of death.

Although several mechanisms have been postulated to explain the synergism between the influenza virus and bacterial pathogens (Hament, et al., J. L. (1999) FEMS Immunol. Med. Microbiol., 26: 189-195) the interaction remains poorly understood. Elucidation of the cellular and molecular basis of the underlying pathogenic mechanisms would likely provide targets for drugs or vaccines that may impact the serious morbidity resulting from influenza infections that are complicated by bacterial infections that occur concurrently with, or as a sequela of the flu. Thus, there is a need for prophylactic and treatment strategies designed to inhibit synergistic mechanisms of viral and bacterial pathogenesis.

### SUMMARY OF THE INVENTION

The invention provides a novel use for neuraminidase inhibitors in chemoprophylactic and treatment methods for the prevention, attenuation and treatment of bacterial infections that may occur in association with, or as a sequela of, viral influenza. The chemoprophylactic methods of the invention are particularly suitable for the prevention of secondary bacterial infections, such as, but not limited to infections of the lower respiratory tract (e.g., pneumonia), middle ear infections (e.g., otitis media) and bacterial sinusitis. More specifically, the chemoprophylactic methods of the invention inhibit mechanisms of viral/bacterial synergism that favor the occurrence of secondary bacterial infections in subjects afflicted with an influenza viral infection. The invention further provides treatment methods that are suitable for treating and/or attenuating the synergistic effects of viral/bacterial pathogenesis that promote the development of severe bacterial infections as sequelae to viral influenza.

In one embodiment, the invention provides a method of preventing secondary bacterial pneumonia in a subject who has been symptomatic for viral influenza for more than 48 hours comprising administering a prophylactically effective amount of a neuraminidase inhibitor.

In a second embodiment, the invention provides a method for achieving chemoprophylaxis of pneumonia in a subject who is at risk of developing bacterial pneumonia as a complication of a viral influenza infection comprising administering a prophylactically effective amount of a neuraminidase inhibitor to the subject. In a particular embodiment the invention provides a post-exposure method of prevention suitable for use in preventing the development of disease in individuals who are at high-risk of developing a complication of influenza infection, such as a lower respiratory tract infection or otitis media. Post-exposure prevention is appropriate for high risk subjects who have been exposed to an individual afflicted with influenza during a time period when the individual was likely to be shedding virus and therefore, was infectious. In an alternative prophylactic embodiment the high risk status of the individual suffices to provide a justification for initiating preventive therapy in an individual who has been symptomatic for at least 48 hours, particularly during periods of time when there is a high incident of influenza in the community.

In a third embodiment, the invention discloses and claims a method for attenuating the pathogenic consequences of a secondary bacterial infection in a subject infected with an influenza virus comprising administering to the subject an amount of a neuraminidase inhibitor effective to prevent the pathogenic synergism between an influenza virus and bacterial pathogen that is known to favor the establishment of severe bacterial infection. In a particular embodiment the administration of a neuraminidase inhibitor confines a pneumococal infection of the lower respiratory tract to a focal (e.g. lobar pneumonia) process as opposed to a severe infection that is disseminated throughout the subject's lung tissue. In an alernative embodiment the invention provides a method for attenuating a secondary bacterial infection of the respiratory tract, such as bacterial sinusitis otitis media, caused by an organism selected from the group consisting of Streptococcus pneumoniae, Haemophilus influenzae, Mycoplasma species and Moraxella catarrhalis.

In a fourth embodiment, the invention provides a method of treating pneumonia in a subject afflicted concurrently with viral influenza and bacterial pneumonia caused by Streptococcus pneumoniae comprising administering a therapeutically effective amount of a neuraminidase inhibitor in combination with a therapeutically effective amount of an antibiotic.

In a fifth embodiment the invention also provides a method of treating a secondary bacterial infection (e.g., walking pneumonia, otitis media, bacterial sinusitis) in a subject who acquires the bacterial infection as a sequelae to a viral influenza infection comprising administering a composition comprising a therapeutically effective amount of a neuraminidase inhibitor in combination with a therapeutically effective amount of at least one antibiotic.

The treatment methods disclosed and claimed herein have been shown to be effective in animal studies, and in light of the established efficacy and tolerability of neuraminidase inhibitors in humans are predicted to be particularly appropriate for use in the treatment of children and high-risk patients in need of relief from concurrent influenza and bacterial infection of either the lower respiratory tract (e.g., pneumonia), the middle ear (otitis media) or the sinus cavities.

The treatment methods of the invention are exemplified by the use of oseltamivir phosphate. However, it is to be understood that other neuraminidase inhibitors can also be used in the methods of the invention, including, for example, zanamivir and RJW-270201 (BCX-1812). According to the methods of the invention disclosed herein the neuraminidase inhibitor and the antibiotic can be administered, simultaneously or sequentially by the same or different routes, depending upon the recommended prescribing practice associated with each of the drugs.

Some of the treatment methods of the invention are suitable for use in treating patients who manifest clinical indicators, and are symptomatic for concurrent viral influenza and bacterial pneumonia infections (e.g. complicated influenza). A skilled practitioner will acknowledge that there are numerous clinical indicators that will be indicative of concurrent infection, or the subsequent development of bacterial pneumonia as a sequela to the flu. Examples of such clinical indicators are difficulty breathing accompanied by a chest examination that indicates rales, consolidation on chest x-ray, and at least one indicator selected from the group consisting of: fever; high white blood cell count and a productive cough. As used herein the term “productive cough” refers to a cough which removes sputum from the respiratory tract.

### BREIF DESCRIPTION OF THE DRAWINGS

FIG. 1. Groups of mice were weighed daily after infection on day 0, with either S. pneumoniae strain D39 (Pneumococcus), and/or influenza virus A/PR/8/34, or exposure to diluent as a mock control. Error bars represent the standard deviation between groups of four mice.

FIG. 2. Mice were infected with S. pneumoniae strain D39 (Pneumococcus) alone on day 0 or dual infected with Pneumococcus and influenza virus A/PR/8/34 either simultaneously on day 0; or sequentially by administering the A/PR/8/34 virus on day 0 and the Pneumococcus on day 7. Blood cultures were collected 24 h after infection with Pneumococcus and quantitated. Titers beyond the limits of the assay are expressed as either >7 if greater than 1×10⁷ CFU/ml, or <2 if less than 100 CFU/ml, respectively.

FIG. 3. Chemoprophylaxis with oseltamivir prolongs survival of mice sequentially infected with influenza virus on day 0 followed 7 days later with a pneumococcal challenge. Oseltamivir chemoprophylaxis was initiated on day 0 at a dose of 10 mg/kg/day and continued for 5 days until the pneumococcal challenge. (p<0.005 by Mantel-Cox Chi Squared test). Instead of oseltamivir, control mice received 100 ul of distilled de-ionized water on the same schedule.

FIG. 4. Oseltamivir treatment of mice infected with influenza virus 48 hrs prior to the administration of a neuraminidase inhibitor and pneumococcal challenge prolongs survival. (p<0.005 by Mantel-Cox Chi Squared test). Oseltamivir chemoprophylaxis was initiated 48 hours after influenza infection at a dose of 10 mg/kg/day and continued for 5 days until the pneumococcal challenge. Instead of oseltamivir, control mice received 100 μl of distilled de-ionized water on the same schedule.

### DETAILED DESCRIPTION OF THE INVENTION

The invention provides a novel use for neuraminidase inhibitors in prophylactic and treatment methods for the prevention, attenuation and treatment of bacterial infections that may occur in association with or as a sequela of viral influenza infections. Suitable neuraminidase inhibitors for use in any of the methods of the invention include, but are not limited to, oseltamivir phosphate (GS4104, Tamiflu®; Roche/Gilead), zanamivir (GG167, Relenza®&; GlaxoSmithKline) and RJW-270201 (BCX-1812, BioCryst). To date, all human clinical trials evaluating the antiviral effects of neuraminidase inhibitors have been performed among patients with uncomplicated influenza and there have been no controlled studies on the efficacy of neuraminidase inhibitors for the chemoprophylaxis, attenuation, or treatment of bacterial infections that occur either concurrently with, or as a sequela of, the flu.

Influenza viruses belong to the Orthomyxoviridae family and are enveloped single-stranded RNA viruses, that occur in three distinct antigenic types (i.e., A, B and C). Types A and B are responsible for epidemics, while type C causes mild respiratory illness. The lipid bilyer coating the influenza virus contains specific hemagglutinin (H) and neuraminidase (N) protein spikes that define distinct viral subtypes. The role of the hemagglutinin is to initiate infection of a host cell by binding to sialic acid residues present on cell surface molecules of a respiratory epithelial cell. The host cell then encloses the virus in an endosome, and the viral and cell membranes subsequently fuse. At a later stage of infection the neuraminidase functions to cleave the bonds between the nascent (newly replicated) viral particles which emerge from the infected host cell coated with a lipid bilayer acquired from the host cell plasma membrane. In the absence of an active neuraminidase, the nascent virions will aggregate at the host cell membrane due to a binding interaction between the viral hemagglutin glycoprotein and host cell surface carbohydrates.

Neuraminidase is an essential enzyme for the replication of influenza virus and it has been described as “molecular scissors” which cut the nascent viruses free. More specifically, the neuraminidase enzyme cleaves terminal neuraminic (sialic) acid residues from carbohydrate moieties on host epithelial cell membrane proteins, and on viral envelope glycoprotein spikes of newly synthesized virions. Generally speaking, neuraminidase enables the release of influenza virions from infected cells, promotes the dissemination of virus within the respiratory tract, and may also reduce the ability of respiratory mucus to inactivate the virus. Inhibition of the neuraminidase enzyme promotes the aggregation of viral particles on the surface of infected cells and effectively interrupts the replicative cycle of the virus.

Neuraminidase inhibitors are analogues of sialic acid, that represent a new class of second-generation anti-viral agents that show efficacy against both influenza A and B viruses. These agents interact with a common region of the active site located in a central cleft that is conserved among all Type A and Type B influenza viruses studied to date despite wide variation in other regions of the enzyme. Neuraminidase inhibitors have been referred to as “plug drugs” and their proposed mechanism of action is to block the active site of the neuraminidase enzyme which effectively leaves uncleaved sialic acid residues on the surface of the host cells and viral envelopes. In the presence of a neuraminidase inhibitor, viral hemagglutinin binds to the uncleaved sialic residues, resulting in viral aggregation at the host cell surface. The inhibition of viral budding results in overall reduction in the amount of virus that is released from infected cells.

The flu and associated complications, including bacterial pneumonia, are the sixth leading cause of death in the world and the leading infectious cause of death. Influenza spreads from person to person via airborne droplets or respiratory fluids, especially after an infected person sneezes or coughs. Influenza viruses generally enter the body through mucous membranes lining the eyes, nose and mouth. Although the flu is considered to be an infection of the respiratory tract, individuals suffering from the flu usually become acutely ill with high fever, chills, headache, weakness, loss of appetite and aching joints. The typical length of time from when a person is exposed to influenza virus to when symptoms first occur ranges between one and five days, with an average of two days. Adults can be infectious (i.e., shedding virus) starting the day before the onset of symptoms begin until approximately 5 days after the onset of illness. Children can be infectious for longer periods of time. Systemic symptoms include abrupt onset of fever (e.g. usually 100-103 degrees F in an adult and possibly higher in children), chills, headaches, myalgia and malaise.

The suddenness with which these symptoms develop usually aid the health care practitioner in distinguishing between influenza and most other viral respiratory infections, such as the common cold, which are generally characterized by a slower onset of symptoms. Even an uncomplicated case of acute influenza is likely to require days of bed rest, and is associated with a general weakness and malaise that can persist for weeks after the cessation of other symptoms. As used herein the term “symptoms” refers to subjective indicia of illness, or a change in condition that is usually related to a health care practitioner by the afflicted subject. Typically, the fever starts dropping on the second or third day of illness as the respiratory symptoms, become more prominent. Respiratory symptoms include dry cough, pharyngeal pain (sore throat), nasal obstruction/discharge and a dry (e.g., nonproductive) cough.

Epidemiological evidence suggests that there is a pathogenic synergism between the influenza virus and the pneumococcus, which accounts for higher levels of morbidity and mortality during the occurrence of an influenza epidemic. As used herein the term “pathogenic synergism” refers to a process or interaction by which two, or more pathogens, interact with each other to enhance the virulence of one or both of the agents. As used herein the term “virulence” refers to the ability of a pathogenic agent (pathogen) to produce a histopathologic effect or symptomatic illness in a host subject. Influenza infections are known to increase the susceptibility of an infected to particular bacterial infections caused by species of bacterial pathogens such as, the pneumococcus, staphylococcus, mycoplasma, non-group H. influenza, and Moraxella catarrhalis.

S. pneumoniae is the most common cause of bacterial pneumonia and accounts for approximately two-thirds of bacteremic community-acquired pneumonias. Studies of the normal pharyngeal flora of healthy people indicate that 5 to 25% of people are carriers of the pneumococcus, with the highest rates noted for young children and their parents during the winter months.

The development of secondary bacterial pneumonia, most commonly pneumococcal or staphylococca pneumonia, is indicated by the either the persistence or reoccurrence of a fever in the second week after the onset of flu symptoms. Often times when pneumonia occurs as a consequence of the flu, the subject's cough worsens and becomes productive. Crepitant or subcrepitant rales can be detected over the involved lung segments. Pneumococci usually reach the lungs by inhalation or aspiration. The inhaled pathogens subsequently lodge in the bronchioles, where they proliferate and initiate an infection and inflammatory process that begins in the alveolar spaces which consequently fill with fluid and provides a foci of infection.

Accordingly, congestion is the earliest stage of classic, pneumococcal lobar pneumonia. A subject afflicted with pneumococcal pneumonia usually reports a sudden onset that commonly begins with persistent chills, followed by fever (e.g., between 100.4 to 105 degrees F), painful breathing on the affected side (i.e. pleurisy), cough, dyspnea, and sputum production. Although the cough may initially be nonproductive, it often becomes productive with purulent, blood-streaked or rusty sputum.

A presumptive diagnosis of pneumonia can be based on a combination of physical examination and patient history, changes in chest X-ray, more specifically the detection of lobar consolidation, the detection of a bacterial pathogen by Gram-stain examination of sputum, a positive quelling reaction, and/or culture of a bacterial organism from sputum samples. More specifically, the Gram-stain of sputum obtained from a patient afflicted with pneumococcal pneumonia will show gram-positive lancet-shaped diplococci in short chains.

Since 1987, the incidence of drug resistant Streptococcus pneumonia disease (DRSP) has increased in the United States, and studies indicate that up to 30% of S. pneumoniae strains are resistant to penicillin, and many of the penicillin-resistant strains exhibit multidrug resistance. Most resistant strains respond to cefotaxime or ceftriaxone, vancomycin and to some of the newer quinolones, such as levofloxacin, sparfoxacin, grepafloxacin and trovafloxacin.

The invention disclosed and claimed herein is based on the discovery that the administration of a neuraminidase inhibitor, namely oseltamivir phosphate, prevents death from and attenuates the morbidity associated with secondary bacterial pneumonia in a mouse model of lethal synergism between influenza virus and Streptococcus pneumoniae. As demonstrated herein, efficacy can be observed even when the treatment protocol is such that the oseltamivir administration does not inhibit viral replication or improve the symptoms of influenza. The chemoprophylactic and treatment methods disclosed and claimed herein stand in contrast to the current usage of neuraminidase inhibitors as antiviral agents. In fact, conventional antiviral treatment regimens require that administration be initiated within the first 48, and optimally within the first 24, 30 or 36 hours of onset of influenza symptoms.

While Applicants do not intend to be bound by any mechanism of action, it appears as if the underlying mechanism of action accounting for the lethal synergism between the influenza virus and pneumococcus is independent of the neuraminidase inhibitor's ability to inhibit viral budding. Accordingly, effective chemoprophylaxis, attenuation and/or treatment of bacterial infection can be achieved by administration of a neuraminidase inhibitor to a subject that has been symptomatic for more than 36 or 48 hours.

In one embodiment, the invention provides a method of preventing secondary bacterial pneumonia in a subject who has been symptomatic for viral influenza for more than 48 hours comprising administering a prophylactically effective amount of a neuraminidase inhibitor. As used herein the term “prophylactically effective amount” is intended to mean that amount of a drug (neuraminidase inhibitor) that will prevent or reduce the risk of occurrence of an undesirable biological or medical event that is sought to be prevented in a tissue, a system, or subject by a doctor or other health care practitioner.

As used herein, the term “neuraminidase inhibitor” includes agents capable of inhibiting at least one enzymatic activity that typifies a neuraminidase protein obtained from a virulent strain of a Type A or Type B influenza virion for a time sufficient to confer either a prophylactic or therapeutic benefit to the subject to whom it is administered. The prophylactic and treatment protocols of the invention contemplate the use of a neuraminidase inhibitor in combination with other agents such as an analgesic, for pleuritic pain, or a decongestant to provide relief for discomfort associated with other symptoms of the underlying viral infection.

In a second embodiment, the invention provides a method for achieving chemoprophylaxis of pneumonia in a subject who is at risk of developing bacterial pneumonia as a complication of a viral influenza infection comprising administering a prophylactically effective amount of a neuraminidase inhibitor to the subject. In a particular embodiment the invention provides a post-exposure method of prevention. Post-exposure prevention is appropriate for high risk individuals who have been exposed to an individual afflicted with influenza. The invention provides that for this aspect of the invention the neuraminidase inhibitor should be administered within two weeks of the subject's exposure, and preferably within four (4) days. In an alternative prophylactic embodiment the high risk status of the individual suffices to provide a sufficient justification for preventive therapy, particularly if there is a high incidence of influenza in the community.

Generally speaking, the prophylactic methods of the invention are particularly suitable for individuals who are identified as at high risk for developing a secondary bacterial infection associated with the flu, including individuals who are at least 50 years old, individuals who reside in chronic care facilities, subjects who have chronic disorders of the pulmonary or cardiovascular system, patients who required regular medical follow-up or hospitalization during the preceding year because of chronic metabolic diseases (including diabetes mellitus), renal dysfunction, hemoglobinopathies, or immunosuppression (including immunosuppression caused by medications or by human immunodeficiency [HIV] virus); children less than 14 years of age, patients between 6 months and 18 years of age who are receiving long-term aspirin therapy, and women who will be in the second or third trimester of pregnancy during the influenza season. More specifically, it is contemplated that both the chemoprophylactic and treatment methods of the invention are suitable for the prevention, attenuation and treatment of bacterial infections in subjects older than 1 years of age and less than 14 yrs of age (i.e., children); young adults who are older than 15 years of age but less than 30 years of age; middle-age adults between the ages of 30 and 50 years of age as well as older subjects, who are known to be an increased risk of developing complications from an influenza infection, including subjects between the ages of 50 and 65, and adults who are older than 65 years of age.

In a third embodiment, the invention discloses and claims a method for attenuating the pathogenic consequences of a secondary bacterial infection in a subject infected with an influenza virus comprising administering to the subject an amount of a neuraminidase inhibitor effective to prevent a pathogenic synergism between an influenza virus and bacterial pathogen that characteristically promotes a severe bacterial infection.

As used herein the term “attenuating” refers to the ability of a drug (e.g., a neuraminidase inhibitor or an antibiotic) to lessen or diminish the virulence of a pathogen. As used herein the term “secondary bacterial infection” refers to a bacterial infection that occurs as a consequence of, or as a sequela to an influenza infection. As used herein the term encompasses, but is not limited to bacterial pneumonia, otitis media, and sinusitis infections that occur as a consequence of a viral influenza infection.

The prophylactic methods of the invention are particularly suitable for the prevention and/or attenuation of secondary bacterial infections such as, but not limited to infections of the lower respiratory tract (e.g., pneumonia), middle ear infections (e.g., otitis media) and bacterial sinusitis. The targeted secondary bacterial infections, may be caused by numerous bacterial pathogens. For example, they may be mediated by at least one organism selected from the group consisting of: Streptococcus pneumoniae; Staphylococcus aureus; Haemophilus influenza, Myoplasma species and Moraxella catarrhalis.

More specifically, the prophylactic methods of the invention inhibit pathogenic mechanisms of viral/bacterial synergism that facilitate the occurrence of secondary bacterial infections in subjects afflicted with an influenza viral infection. Thus, the invention provides a method for inhibiting synergistic pathogenic mechanisms that promote the development of severe bacterial infections as sequela to a viral influenza infection. Generally speaking, the prophylactic methods of the invention can be used to prevent the occurrence of such secondary infections as, but not limited to, bacterial pneumonia, otitis media, and bacterial sinusitis.

Otitis media, is an inflammation of the ear that presents with a rapid onset of signs and symptoms. Acute otitis media occurs when fluid from the ear canal, or eustachian tube, collects in the middle ear and becomes infected. Pathogenic synergism between the influenza virus and bacterial pathogens may promote the occurrence of otitis media in association with, or as a consequence of the flu by impairing eusthachian-tube function and other host defenses, such as damaging the respiratory epithelial cell barrier. Otitis media can manifest with signs or symptoms of acute local and/or systemic illness. The fluid presses on the eardrum, causing the afflicted subject to experience an earache. In addition to ear pain, other clinical indicators and symptoms include otalgia, otorrhea, a bulging red or yellow tympanic membrane, fever, irritability, sleep disturbances and anorexia. The three major pathogens associated with the occurrence of otitis media include: S. pneumoniae, H. influenza and Moraxella catarrhalis.

While not wishing to be bound by any particular theory, the inventor speculates that one possible mechanism of pathogenic synergy may result from the effects of influenza-virus mediated cleavage of terminal sialic acid from epithelial cells lining the subject's lungs. Thus, in a particular example of this embodiment the invention provides a method for inhibiting a pathogenic synergism that is likely to be dependent upon viral neuraminidase-mediated exposure of pneumococcal receptors on lung epithelial cells. For example, in a particular embodiment, the invention discloses a method in which inhibition of the pathogenic synergism between influenza virus and Steptococcus pneumoniae bacteria results in a lower respiratory infection (e.g., pneumonia) that is restricted to a focal infection, that is characteristic of primary pneumococcal pneumonia, as opposed to a serious secondary bacterial infection that disseminates throughout the lung tissue.

In a fourth embodiment, the invention provides a method of treating pneumonia in a subject afflicted concurrently with viral influenza and bacterial pneumonia comprising administering a therapeutically effective amount of a neuraminidase inhibitor in combination with a therapeutically effective amount of an antibiotic. In a particular embodiment, the invention provides a method of treating secondary Pneumococcal pneumonia.

In a fifth embodiment the invention also provides a method of treating secondary bacterial infection (e.g., walking pneumonia, otitis media, sinusitis) caused by Streptococcus pneumoniae in a subject who acquires the bacterial infection as a sequelae to a viral influenza infection comprising administering a composition comprising a therapeutically effective amount of a neuraminidase inhibitor in combination with a therapeutically effective amount of at least one antibiotic.

As used herein the term “concurrently” refers to a condition in which an individual or subject has both a viral influenza infection and an associated bacterial infection at the same time. It is to be understood that the term does not indicate that the conditions have to be acquired simultaneously or have a coextensive durations. Associated bacterial infections are infections that occur as a result of the colonization of a tissue that is associated with the respiratory tract, including the ears, nose, sinuses, throat and lungs.

As used herein, the term “therapeutically effective amount” is intended to mean that amount of a drug (e.g., neuraminidase inhibitor or antibiotic) that will elicit the biological or medical response (e.g., treatment and/or attenuation of an infection or inflammatory process mediated by a pathogen) of a tissue, a system, or subject that is being sought by a doctor or other health care practitioner.

The treatment methods disclosed and claimed herein have been shown to be effective in animal studies, and in light of the established efficacy and tolerability of neuraminidase inhibitors in humans are predicted to be appropriate for use in the treatment of children and high-risk patients in need of relief as a result of concurrent influenza virus and a bacterial infection of the lower respiratory tract (e.g., pneumonia).

The methods of the invention are exemplified by the use of oseltamivir phosphate RO-64 0795 (GS4104; Tamiflu®; Roche/Gilead) which is an ethyl ester prodrug that requires ester hydrolysis for conversion to the active form, oseltamivir carboxylate (GS407). As used herein the term prodrug refers to a compound that is transformed in vivo to yield a biologically active agent, for example by enzymatic hydrolysis. More specifically, as used herein the term encompasses neuraminidase inhibitors that requiring processing in vivo in order to be activated. It is to be understood that other neuraminidase inhibitors could also be used in the embodiments of the invention disclosed and claimed herein. For example, zanamivir (GG167, Relenza®; Glaxo Wellcome) or RJW-270201 (BCX-1812) could also be used.

Zanamivir is approved for treatment of uncomplicated acute influenza in person over 12 years of age who have been symptomatic for no more than 2 days, it is most effective if administered within 30-36 hrs of symptom onset. Zanamivir is poorly absorbed in the gastroinstestinal tract and is formulated as a dry powder for inhalation. Most of the drug is deposited in the throat; approx. 20% of the inhaled drug reaches the lungs and the drug has a half-life of 2.5 to 5 hrs.

Oseltamivir is approved for treatment of uncomplicated influenza in adults over the age of 18 years who have been symptomatic for no more than 2 days. Oseltamivir is a long-acting oral agent with a systemic bioavailability of 80% and a half-life of 6 to 10 hours. The proposed mechanism of action of oseltamivir is inhibition of influenza virus neuraminidase and prevention of viral budding from infected cells. Oseltamivir is indicated for treatment of uncomplicated acute influenza in adults who have been symptomatic for less than 48 (i.e., 60 or 40) hours. The recommended oral dose of oseltamivir is 75 mg twice daily for 5 days. To date there is no evidence for efficacy of oseltamivir in any illness caused by pathogens other than influenza viruses Types A and B.

Suitable antibiotics that could be coadministered in combination with a neuraminidase inhibitor include, but are not limited to, at least one antibiotic selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime,trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine. The neuraminidase inhibitor and the antibiotic can be administered simultaneously or sequentially by the same or different routes, depending upon the recommended prescribing practice associated with each of the drugs.

For example, oseltamivir phosphate can be administered as an oral systemic agent in the form of a prodrug in combination with at least one antibiotic selected from the group consisting of, but not limited to, ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid could be administered intravenously. Alternatively, zanamivir can be administered topically, for example, intranasally or by oral inhalation using a diskhaler and an antibiotic selected from the group consisting of, but not limited to amoxicillin, amoxicillin/clavulinic acid (Augmentin), cefuroxime, trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, cephradine, could be administered by mouth or intravenously.

It is well-known that due to the widespread use, and frequent over-prescribing of antibiotics, there is an increasing incidence of bacterial pathogens acquiring drug-resistance, including mutlidrug resistance. In other words, bacterial pathogens that are typically susceptible to (i.e., inhibited or killed) to a particular antibiotic are no longer susceptible. Accordingly, the use of a neuraminidase inhibitor to attenuate the virulence of a bacterial pathogen, or to treat a concurrent infection could decrease the occurrence of drug-resistance due to milder bacterial infections that lower the overall colonization that occurs when a bacterial pathogen is establishing an infection in a particular site, or possible by lower the dosage or length of time that an antibiotic is required to treat the infection.

In some embodiments of the invention, the disclosed treatment methods of the invention are suitable for use in patients who manifest clinical indicators of concurrent viral influenza and bacterial pneumonia infections. As used herein the term “pneumonia” refers to an infection and inflammation of the lungs, in which the alveoli and bronchioles of the lungs become blocked with a fibrous exudate. As used herein the term “lobar pneumonia” refers to a severe bacterial infection comprising a focal process that is confined to one or more of the five major lobes of the lungs, that if left untreated will result in consolidation of lung tissues.

As used herein the term “clinical indicators” refers to objective information that can be obtained from a medical history or physical examination of a subject, without the aid of laboratory test of x-ray films. For example, a physical exam that results in the detection of a fever, or the detection or rales during a physical examination of the chest constitute clinical indicators. A skilled practitioner will acknowledge that there are numerous clinical indicators that will be indicative of concurrent infection such as difficulty breathing accompanied by a chest examination that indicates rales, consolidation on chest x-ray, and at least one indicator selected from the group consisting of: fever; high white blood cell count; and a cough.

As used herein the term “rales” occurs to a clinical indicator defined as an abnormal respiratory sound characterized by discontinuous bubbling noises that can be heard on auscultation of the chest during inspiration. Auscultation refers to the practice of listening for sounds within the body to evaluate the condition of an organ or to detect a fetal heart beat. Auscultation may be performed directly with the unaided ear, but is most commonly performed with a stethoscope to determine the frequency, intensity, duration, and quality of the sounds. As used herein the term “consolidation” as it is used to refer to lung tissue refers to a condition in which the lung tissue looses its elasticity and becomes solid. The detection of consolidated lung tissue is indicative of pneumonia. A skilled medical practitioner can identify the presence of consolidated lung tissue on a chest x-ray.

The discovery of the efficacy of neuraminidase inhibitors in the prevention and/or treatment of bacterial infections were observed using a murine model of pneumonia and sepsis designed for the purpose of elucidating and studying the pathogenic mechanisms that contribute to the lethal synergism that is characteristic of the murine model disclosed herein. Although, the consequences of the synergistic interaction of viral/pneumococcus mechanisms contributing to the development of otitis media have been studied in the chinchilla model (S. Giebink, (1999) Microb. Drug Resist.: 5 57-72), a small animal model of influenza infection complicated by bacterial pneumonia has not been previously described. This model finds utility in determining the mechanisms involved in pathogenic interactions between influenza virus and pneumococcus.

Several exemplary dosing regimens are contemplated, depending upon the condition being prevented or treated and the stage to which the underlying viral infection and the associated bacterial infection have progressed. For chemoprophylactic purposes with respect to pneumococcal pneumonia, for example, a low chronic dose sufficient to inhibit the removal of sialic acid from the carbohydrate surface moieties of epithelial cells lining the respiratory tract of a subject who has been symptomatic for a viral influenza infection for at least 48 hours.

An exemplary dose of a neuraminidase inhibitor that is appropriate for use in the treatment methods of the invention is about 75 mg (ranging between about 25 and about 100 mg) by mouth twice a day of oseltamivir for adults weighing more than 40 kg, or 4mg/kg by mouth twice a day for children. An exemplary dose of Zanamivir for use in the treatment methods of the invention includes 1 metered dose twice a day administered by diskhaler. Acute periods of administration for a period of time ranging between 5 and 15 days (e.g., 5, 7, 10 12 or 14 days) would be appropriate for use in the treatment methods disclosed and claimed herein.

Low doses, such as 10 mg daily (oseltamivir) or 1 mg daily (zanamivir) are contemplated for use in the chemoprophylactic methods disclosed and claimed herein. An exemplary dose range for oseltamivir comprises doses ranging from about 75 mg (or 2 mg/kg) once or twice daily to about 150 mg (or 4 mg/kg) once or twice daily. Administration for an extended or chronic period of time, for example 5, 7, 10, 12, or 14 days or longer, such as for a period of 2, 3, 4, 5, 6, 7, 8, 10, 12 14, or 16 weeks is contemplated. An exemplary dose range of zanamivir ranging from about 10 mg (1 metered dose) once or twice daily to about 20 mg (2 metered doses) administered once or twice daily administered for a chronic period of time is also contemplated.

Relatively higher doses, such as 500 mg twice daily (oseltamivir) or 96 mg once daily (zanamivir) may be required to attenuate the severity of a secondary bacterial infection, the treatment of which should be initiated soon after the subject becomes symptomatic for the bacterial infection. No adverse side effects (e.g., nausea) should occur as a consequence of any of the dosing regimens that are employed to practice the methods of the invention described herein.

The prophylactic and therapeutic compositions of the invention can be administered using an amount (e.g., dose) and any route of administration effective for preventing, attenuating or treating a bacterial infection. The treatment protocols of the invention contemplate the use of a neuraminidase inhibitor in combination with an antibiotic. The choice of an appropriate antibiotic will depend upon the pathogen suspected of causing the infection and the effected organ or tissue in which the infection occurs. Suitable antibiotic doses will be well-known to health care practitioners. The exact dosage of neuraminidase inhibitor and antibiotic required to practice either a chemoprophylactic or therapeutic method of the invention will vary from subject to subject, depending on such considerations as the, age and general condition of the subject, the severity of and stage of the underlying viral infection, the particular neuraminidase inhibitor and antibiotic selected for use, the mode of administration and the like. Determination of suitable dosing regimens is well within the skill of a qualified health care practitioner.

### EXAMPLES

The following examples are presented by way of illustration, not by way of limitation:

Materials and Methods

Infectious Agents

Mouse adapted influenza virus A/Puerto Rico/8/34 (HlN1), hereafter referred to as PR8, was grown in Madin-Darby canine kidney (MDCK) cells from stock from the influenza virus repository at St. Jude Children's Research Hospital. The dose of PR8 lethal for 50% of infected mice (MLD₅₀) was the equivalent of 3000 TCID₅₀ (the dose infectious for 50% of MDCK tissue culture wells) and 140 MID₅₀ (the dose infectious for 50% of mice).

S. pneumoniae D39, a type 2 encapsulated strain, was obtained from the collection of Elaine Tuomanen, St. Jude Children's Research Hospital, Memphis, Tenn., and grown in Todd Hewitt broth (Difco Laboratories, Detroit, Mich., USA). The MLD₅₀ for pneumococcus in mice was equivalent to 5×10⁵ CFU on tryptic soy agar (Difco Laboratories) supplemented with 3% v/v sheep erythrocytes.

Mice

Eight- to ten-week-old female Balb/cByJ mice (Jackson Laboratory, Bar Harbor, Me.) were maintained in a Biosafety Level 2 facility in the Animal Resource Center at St. Jude Children's Research Hospital. All experimental procedures were approved by the Animal Care and Use Committee prior to study and were done under general anesthesia with methoxyflurane (Pittman-Moore, Mundelein, Ill., USA).

Infection

Depending upon the experimental protocol (e.g., infection only or treatment protocol) mice were infected intranasally with 100 to 1500 infectious units (TCID₅₀=tissue culture infectious dose 50%) of the above described influenza virus strain PR8 and 100 to 100,000 infectious units (CFU=colony forming units) of D39 pneumoccocus.

Generally speaking, the mice assigned to one of the infection only protocols described in Examples 1-3 were infected with 1500 TCID₅₀ PR8 and 100,000 CFU D39; while mice assigned to one of the oseltamivir treatment protocols described in Examples 4-5 were infected with 100 TCID₅₀ PR8 and 100 CFU D39.

Neuraminidase Inhibitor-Oseltamivir

Oseltamivir is administered as a prodrug and the absorbed GS4104 (RO-64 0796) is metabolized primarily by the action of hepatic esterases, to GS4071 (oseltamivir carboxylate), which is the active neuraminidase inhibitor, primarily by the action of hepatic esterases.

Chemoprophylactic Protocol

Chemoprophylaxis with oseltamivir was initiated on day 0, 4 hours prior to infection with influenza virus. The mice received a dose of 10 mg/kg/day divided BID of the oseltamivir prodrug RO-64-0796 (Roche, Welwyn Garden City, England) suspended in a volume of 100 microliters of distilled deionized water. Chemoprophylaxis was administered for 5 consecutive days, until the pneumococcal challenge on day 7.

A group of control mice were administered 100 microliters of distilled, deionized water BID for 5 days.

Treatment Protocol

The treated mice received the same dose of neuraminidase inhibitor that was administered in to the mice in the chemoprophylactic study (10 mg/kg/day divided BID of the oseltamivir prodrug RO-64-0796). However treatment was not initiated until 48 hours had elapsed from the time of infection with influenza virus. Treatment was continued for 5 days until the pneumococcal challenge on day 7.

Blood Cultures

Approximately 500 μl blood was obtained from mice via retroorbital puncture with polished, sterile, glass Pasteur pipettes 24 h following infection with pneumococcus and transferred into Isolator 1.5 microbial tubes (Wampole Laboratories, Cranbury, N.J., USA).

Quantitation of colony counts by the Isolator 1.5 system was done by tenfold dilutions on tryptic soy agar plates supplemented with 3% v/v sheep erythrocytes. The assay could quantitate colony counts between 100 and 10⁷ CFU/ml blood.

Determination of Mean Survival Day

A mean survival day (MSD) was calculated using the following formula

MSD=[f(d−1)]/N

where f is the number of mice recorded dead on day d (survivors on day 21 were included in f for that day), and N is the number of mice in a group.

### EXAMPLES

### Example 1

### Infection with Influenza virus and/or Pneumoccous

100 μl volumes (50 μl in each nostril) of in phosphate buffered saline (PBS) comprising either no infectious agent (negative control group); 1500 TCID50 PR8; 100,000 CFU D39 pneumococcus; or 1500 TCID50 PR8 and 100,000 CFU D39 pneumococcus was administered intranasally to 4 group of mice.

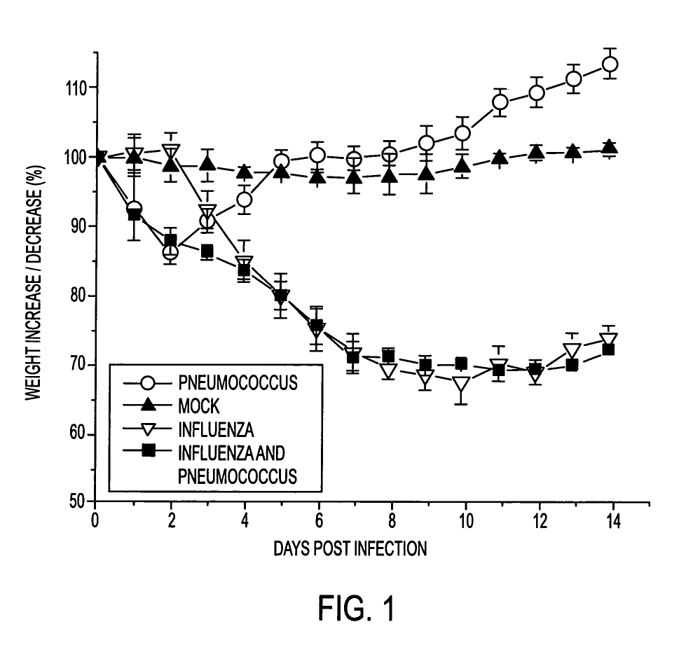

Results: Mice infected with PR8 initially showed no disease signs, but after 3-4 days developed clinical indications/symptoms of infection that were manifest as ruffled fur, decreased activity, anorexia, huddling, hunched posture, and shivering. Weight loss was noted after 2 days and peaked at 7-10 days with approximately 30% weight loss (FIG. 1) after which time surviving mice gradually recovered.

Mice infected with pneumococcus were clinically ill within 24 h as evidenced by decreased movement, shivering, huddling, hunched posture, and closed eyes. Weight loss occurred between days 1 and 3 but mice recovered quickly by 5 days post infection (p.i.).

Mice assigned to the simultaneous dual infection (influenza and pneumococcus) group exhibited gradual weight loss and mortality, commensurate with expectations for an additive process. The simultaneously infected mice exhibited signs of each infection with rapid onset of clinical illness and gradual recovery after a weight nadir at 7-10 days (FIG. 1). The negative control (mock infected) animals exhibited no clinical symptoms and no change in weight. The data indicate that mice can be infected with both influenza virus and pneumococcus and that simultaneous infection results in morbidity and mortality consistent with what would be expected from an additive process or interaction.

### Example 2

### Sequential Infection with influenza Followed by pneumococcocci Mediates Lethal Synergy

In order to determine if the lethal synergy observed in the simultaneous dual infection model described in Example 1 was due to influenza predisposing the mice to a more severe pneumococcal infection, an experiment was performed in which the pneumococcal challenge was delayed for 7 days after infection with the influenza virus. Groups of 8 mice were divided into 5 experimental groups. Group 1 mice were infected on day 0 with PR8 influenza virus; Group 2 mice were infected on day 0 with pneumococcus D39; Group 3 mice were simultaneously dual infected with PR8 and pneumococcus on day 0; Group 4 mice were dual infected sequentially with PR8 virus being administered on day 0 and pneumococcus on day 7; Group 5 mice were the negative control animals and received an equivalent volume of PB5 comprising no pathogenic agent. All of the mice assigned to experimental groups were infected with doses described in Example 1. Mice were followed for mortality for 21 days, and gross mortality and the MSD were determined.

Results: Mice infected with either pathogen alone (Groups 1 and 2) or simultaneously with both pathogens (Group 3) manifested clinical indicators of infection and a mortality rate of 25-50%. Generally speaking, mice exhibited a clinical illness of 9-15 days. The average MSD for these groups ranged between 14 and 16 days.

All of the mice in the negative control group survived with a maximum attainable MSD of 20 days, and exhibited no clinical indicators of infection. All of the Group 4 mice that were sequentially infected with influenza virus on day 0, and challenged on day 7 with pneumococcus, uniformly died within 24 hours of the challenge. These data indicate that sequential infection with influenza virus followed by a pneumococcal challenge on day 7 mediates a lethal synergy that produces 100% lethality in less than 24 hours.

TABLE 1
Infection with pneumococcus following influenza is lethal in mice

| | | | Mortality |
|---|---|---|---|
| Group | Pathogen(s) | MSD | [No. Surviving/Total(%)] |
| 1 | Influenza | 14.1 | 4/8 (50) |
| 2 | Pneumococcus | 15.6 | 6/8 (75) |
| 3 | Influenza and Pneumococcus | 14.3 | 4/8 (50) |
| 4 | Influenza then Pneumococcus | 0 | 0/8 (0) |
| 5 | Mock infected | 20.0 | 8/8 (100) |

### Example 3

### Dual Infected Mice are Highly Bacteremic

To determine whether the mortality observed in the sequential dual infection model was attributed to bacterial sepsis, the experiment described in examples was repeated and blood cultures were obtained 24 hours after pneumococcal challenge. Briefly, groups of eight mice were infected with either pneumococcus, PR8 and pneumococcus together, or PR8 followed 7 days later by pneumococcus. Blood cultures were obtained 18-24 h after infection with pneumococcus and quantitated.

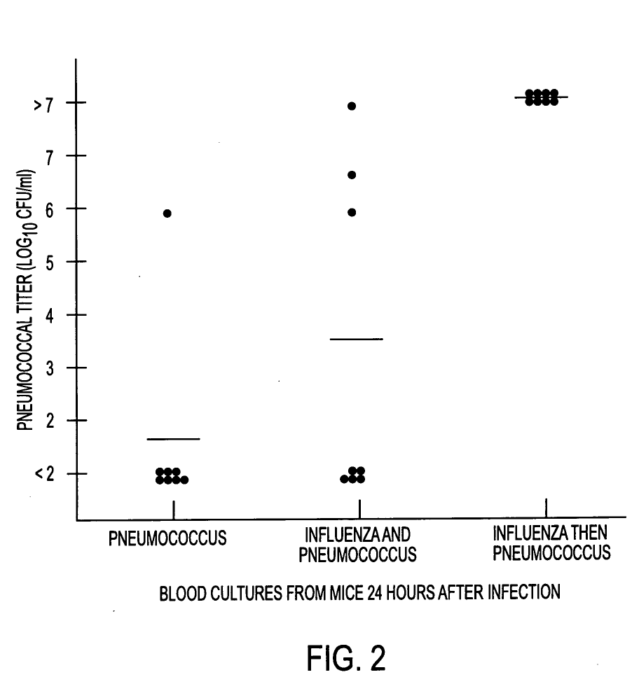

Results: All mice sequentially infected with pneumococcus 7 days after infection with PR8 were highly bacteremic above the limits of the assay as performed. In contrast, only one mouse had bacteremic in the group infected with pneumococcus alone while the group receiving both pathogens simultaneously had intermediate levels of sepsis (FIG. 2). The blood culture data indicates that the sequentially dual-infected mice were highly bacteremic. This data suggests that the rapid demise of the sequentially infected mice is attributed to overwhelming sepsis.

Additional experiments performed in the sequential dual infection model indicated that reduction of the doses of each of the pathogens used to infect the mice elicit similar results with a 100% mortality rate following an underlying clinicopathologic course of disease that is more consistent with pneumonia than with sepsis (McCullers et al., manuscript in preparation).

### Example 4

### Chemoprophylaxis Study

Two groups of mice were infected intranasally (i.n.) with 100 TCID₅₀ of mouse adapted influenza A/PR/8/34 (HIN1) on day 0. One group (prophylaxis group) was begun on oseltamivir (as the prodrug RO-64-0796 provided by Roche) for 5 days at a dose of 10 mg/kg/day OG divided BID in a volume of 100 μl suspended in distilled, de-ionized water 4 hours prior to infection with the influenza virus. The control group was given 100 ∞l of distilled, de-ionized water BID for 5 days on the same administration schedule. Seven days after infection with influenza virus, the mice were challenged i.n. with 100 CFU D39. The mice were followed for morbidity (weight loss) and mortality for 21 days. Some of the mice from each group were sacrificed on day 3 (peak titers) and day 7 (prior to challenge with pneumococcus) and viral lung titers were determined.

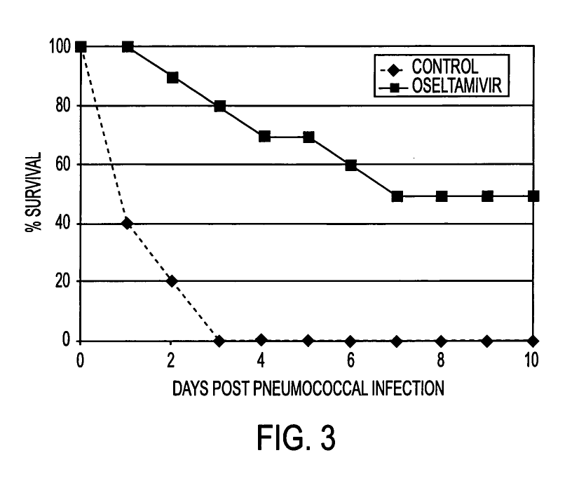

Results: Survival was significantly prolonged (p<0.005 by Mantel-Cox Chi-Squared test on the Kaplan-Meier survival data) in the group of mice receiving oseltamivir (5/10 survived vs. 0/10—see FIG. 3). Median survival was <1 day for the control group and 7 days for the prophylaxis group. Surviving mice in the control group had lost 9.5% of their day 7 weight in 48 hours while surviving mice in the prophylaxis group had lost 7.5% of their day 7 weight. Mean viral titers at day 3 were 6.8 in the control group versus 4.3 in the prophylaxis group (measured as the log₁₀/ml of the dose infectious for 50% of MDCK tissue culture wells—the TCID₅₀). Mean viral titers at day 7 (prior to challenge with pneumococcus) were 6.92 in the control group versus 3.84 in the prophylaxis group (Table 2).

TABLE 2
Viral lung titers and weight loss

| | | | Control | Oseltamivir |
|---|---|---|---|---|
| | | | Group | Group |
| | Control | Oseltamivir | Weight Loss | Weight Loss |
| | Group Mean | Group Mean | (% of | (% of |
| | Titer (log₁₀) | Titer (log₁₀) | Baseline) | Baseline) |
| Propylaxis | | | | |
| Study | | | | |
| Day 3 | 6.80 | 4.32 | 1.5 | 4.0 |
| Day 7 | 6.92 | 3.84 | 24 | 8.7 |
| Treatment | | | | |
| Study | | | | |
| Day 3 | 7.16 | 6.84 | 6.1 | 7.6 |
| Day 7 | 6.14 | 5.42 | 28 | 23 |
| Day 8 | 9.00 | 7.08 | | |
| Bacterial | | | | |
| Titer | | | | |

### Example 5

### Treatment Study

A second sequential dual infection study was performed using the same infection protocol and schedule that was outlined in the above-described prophylaxis study, except that oseltamivir administration was not begun until 48 hours after influenza infection. Oseltamivir treatment was continued until the challenge with pneumococcus on day 7. In addition, mice from each group were sacrificed on day 8 (24 hours after pneumococcal challenge) to titer bacterial counts in the lung and to send lung tissue samples for histopathology.

It should be noted that the oseltamivir groups from both studies cannot be directly compared because the mice were different sizes/ages. More specifically, the mice in the “treatment” study were about 2 weeks younger and 10% lighter.

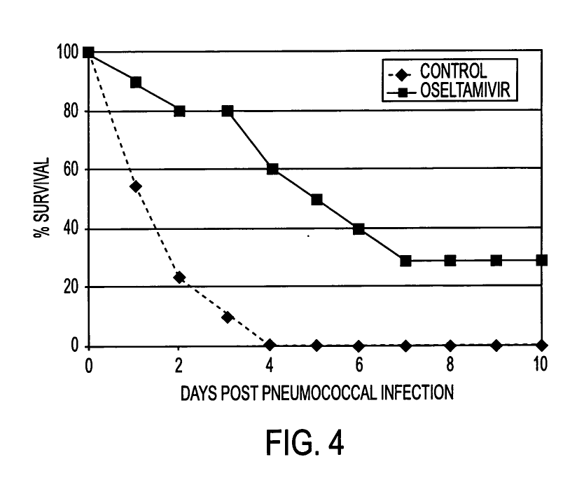

Results: Survival was significantly prolonged (p<0.01 by Mantel-Cox Chi-Squared test on the Kaplan-Meier survival data) in the group of mice receiving oseltamivir (3/10 survived vs. 0/9—see FIG. 4). The above-noted difference in the age and starting weight of the mice may account for the higher mortality rate in the oseltamivir-treated group of the treatment study.

Median survival was 1 day for the control group and 5 days for the prophylaxis group. Surviving mice in the control group had lost 5% of their day 7 weight in 48 hours while surviving mice in the treatment group had lost 4% of their day 7 weight. Mean viral titers at day 3 were 7.1 in the control group versus 6.8 in the treatment group. Mean viral titers at day 7 were 6.1 in the control group versus 5.4 in the treatment group (Table 1). Mean bacterial lung titers 24 hours after pneumococcal challenge were 9.0 in the control group (measured as log₁₀ CFU/ml) versus 7.1 in the treatment group (Table 1).

The histopathology performed on lung tissue samples obtained from the untreated control group mice indicates that pre-infection with influenza virus leads to a diffuse, severe bacterial infection that is disseminated throughout the tissue of both lungs, as opposed to the focal infection process that typifies a classic primary infection.

In contrast, histopathological examination of lung tissue samples obtained from the lungs of mice treated with oseltamivir evince a lobar bronchopneumonia infection that is confined to defined regions of the lungs. This suggests that the efficacy of neuraminidase inhibitors observed in the chemoprophylaxis and treatment methods disclosed and claimed herein may be attributed to inhibition of neuraminidase-mediated cleavage of terminal sialic acid resides from epithelial cells lining the respiratory tract.

Discussion

In the murine models described herein, infectious doses below the MLD₅₀ of influenza virus, or pneumococcus alone, resulted in partial mortality, with deaths occurring after a progressive clinical illness. Mice that were simultaneously infected with both an influenza virus and pneumococcus exhibited a course of infection and a mortality rate that would be expected for an additive process assuming that each of the pathogens mediate disease by an independent process.

In contrast, when mice infected with influenza virus were substantially challenged 7 days later with pneumococcus, the sequential dual infection demonstrates a lethal synergism that results in rapid (e.g. within 24 hours) and complete mortality. The fact that the observed synergism occurred when influenza infection preceded pneumococcal infection, and not when the viral and bacterial pathogens were administered simultaneously, suggests that some alteration in the mouse engendered by the influenza virus infection predisposes the sequentially dual infected mice to sepsis.

In theory, pathogenic bacteria may interact synergistically with influenza viruses in numerous ways. Particularly relevant possibilities to the model disclosed herein include the possibility that bacterial products may directly, or indirectly, result in a more efficient cleavage of the heamagglutinin glycoprotein of the influenza virus, thereby expanding the apparent tissue tropism or virulence of the influenza virus. Alternatively, pathogenic mechanisms of the virus may function to depress the local immunocompetence of the host, thereby enhancing the pathogenicity and severity of a concurrent, or secondary, bacterial infection. A third possibility is that the consequences of viral replication may, through a variety of mechanisms, provide new, or more efficient, sites for bacterial adherence to host cells thereby increasing the severity of a concurrent or subsequent bacterial infection.

While not wishing to be bound by a particular theory, the inventor believes that the lethal synergism observed in the sequential dual infection model disclosed herein may be attributed to either a suppression of the immune system, a destruction of the respiratory epithelium which expose basement membrane elements that the pneumococcus can adhere to, general debilitation of the mouse, or upregulation of receptors permissive for pneumococcal adherence and invasion as a consequence the host cytokine response to the initial influenza virus infection.

While the inventor acknowledges that it is likely that multiple factors are responsible for the observed synergism, the latter hypothesis (enhanced pneumococcal adherence) is particularly attractive in light of published studies establishing that the PAF receptor can be upregulated in vitro facilitating pneumococcal attachment and invasion following exposure to cytokines (e.g., TNF-alpha and IL-1) known to present during influenza virus infection. Cundell, et al., Nature, (1995) 377:435-438.

It is known that pneumococcus must remove terminal sialic acid from cells to gain access to certain cellular receptors that facilitate bacterial infection. In fact pneumococcus carries its own neuraminidase for this very purpose. One potential mechanism of synergy that is consistent with the data provided herein is that influenza virus is stripping sialic acid off of large portions of the host's lung tissue which exposes receptors for pneumococcus and facilitates bacterial dissemination throughout the lungs of the untreated mice. The characteristic lobar histopathology that typifies primary pneumococcal likely results from the fact that the pneumococcus is confined to a focal area of infection that is defined by limits imposed by the localized region in which the concentration of its own neuraminidase is sufficient to allow the pneumoccocus to adhere to host cells. Accordingly, although a neuraminidase inhibitor may not be able to prevent viral replication in an individual who has been sympotmatic for influenza for more than 48 hours, it may be able to effectively inhibit and/or abrogate the underlying pathogenic mechanism which accounts for the viral/bacterial synergy evidenced herein.

A striking aspect of the disclosed results comes from the observation that administration of a neuraminidase inhibitor to an animal that has been infected with influenza virus for more than 48 hours is efficacious in preventing mortality in dual infected mice. This effect is evident from the viral titer data summarized in Table 2.

Mice infected with oseltamivir in the treatment study had lost 23% of their baseline weight (compared to 28% in the control group) while oseltamivir-treated mice in the prophylaxis study had lost only 9% of their baseline weight (compared to 24% in the control group for that study)—see Table 2. As might be expected, in contrast to the significant differences in viral titers that were observed in the chemoprophylaxis study, there is not a significant difference in the viral titers observed in the oseltamivir treated and the control animals when osetlamivir treatment is delayed until 48 hours after viral infection. The observed differences in the effects on viral titar are consistent with a review of the body weights and observations regarding the well-being (e.g. appearance) of the animals.

The fact that there was a significant improvement in survival in the treatment study, despite similar viral titers and similar weight loss implies that the effect of oseltamivir treatment in the sequentially infected mice is unlikely to be explained by a decreased viral load, or to be a consequence of the oseltamivir-treated mice being “less ill” than the control group.

These observations have important implications for the medical use of oseltamivir and suggests that the 24-48 hour treatment window that is necessary for anti-viral efficacy may not be required for efficacy in the context of preventing, attenuating or treating bacterial infections that occur concurrently with, or as a sequela to the flu. In fact the data disclosed herein suggests that even delayed treatment (in a situation where studies published to date have failed to show any efficacy for neuraminidase inhibitors) can reduce excess morbidity and mortality resulting from flu-associated bacterial infections.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of preventing secondary bacterial pneumonia in a subject who is afflicted with an influenza infection comprising administering a composition comprising a prophylactically effective amount of a neuraminidase inhibitor to a subject who has been symptomatic for viral influenza for more than 48 hours.

2. The method according to claim 1, wherein the neuraminidase inhibitor is selected from the group consisting of: oseltamivir phosphate, zanamivir and RJW-270201 (BCX-1812):

3. The method according to claim 2, wherein the nueraminidase inhibitor is oseltamivir phosphate and the composition is administered orally.

4. A method for achieving chemoprophlyaxis of pneumonia in a subject who is at risk of developing bacterial pneumonia as a complication of a viral influenza infection comprising administering a prophylactically effective amount of a neuraminidase inhibitor to the subject.

5. The method according to claim 4, wherein the neuraminidase inhibitor is administered within 4 days of the subject's exposure to a host afflicted with an influenza viral infection.

6. The method according to claim 4, wherein the subject is a human selected from the group consisting of: an individual who is at least 50 years old, an individual who resides in a chronic care facility, an individual who has a chronic disorder of the pulmonary or cardiovascular system, an individual who has required regular medical follow-up or hospitalization during the preceding year because of chronic metabolic diseases (including diabetes mellitus), renal dysfunction, hemoglobinopathies, or immunosuppression (including immunosuppression caused by medications or by human immunodeficiency [HIV] virus); an individual between 6 months and 18 years in age who is receiving long-term aspirin therapy, an individual less than 14 years of age, and a woman who will be in the second or third trimester of pregnancy during the influenza season.

7. The method according to claim 6, wherein the subject is a human over 65 years of age.

8. The method according to claim 6, wherein the neuraminidase inhibitor is selected from the group consisting of: oseltamivir phosphate, zanamivir and RJW-270201 (BCX-1812).

9. A method for attenuating a secondary infection in a subject infected with an influenza virus comprising administering to the subject an amount of a neuraminidase inhibitor effective to prevent a pathogenic synergism between the virus and a bacterial agent that characteristically promotes a severe bacterial infection wherein the bacterial infection is prevented from disseminating throughout the subject's lung tissue.

10. The method of claim 9, wherein the pathogenic synergism results from the effects of influenza-virus mediated cleavage of terminal sialic acid from epithelial cells lining the subject's lungs.

11. The method according to claim 9, wherein the pathogenic synergism results from viral neuraminidase-mediated exposure of pneumococcal receptors on lung epithelial cells.

12. The method according to claim 9, wherein the secondary infection is a bacterial infection of the lower respiratory tract mediated by an organism selected from the group consisting of: Streptococcus pneumoniae, Staphylococcus aureus, Haemophilus influenzae, Mycoplasma species and Moraxella catarrhalis.

13. The method according to claim 9, wherein effective prevention of pathogeninc synergism restricts a lower respiratory tract infection to a focal process that is characteristic of primary pneumococcal pneumonia as opposed to a severe bacterial infection that disseminates throughout the subject's lung tissue.

14. A method for attenuating a secondary infection in a subject infected with an influenza virus comprising administering to the subject an amount of a neuraminidase inhibitor effective to prevent a pathogenic synergism between the virus and a bacterial agent that characteristically mediates a secondary bacterial infection of the respiratory tract.

15. The method according to claim 14, wherein the secondary infection is a bacterial infection mediated by an organism selected from the group consisting of: Streptococcus pneumoniae, Staphylococcus aureus, Haemophilus influenzae, Mycoplasma species and Moraxella catarrhalis.

16. The method according to claim 15, wherein the secondary bacterial infection is bacterial sinusitis.

17. The method according to claim 15, wherein the secondary infection is otitis media.

18. A method of treating pneumonia in a subject afflicted concurrently with viral influenza and bacterial pneumonia caused by Streptococcus pneumoniae comprising administering a therapeutically effective amount of a neuraminidase inhibitor in combination with a therapeutically effective amount of at least one antibiotic.

19. The method according to claim 18, wherein the subject manifests clinical indicators comprising:
a. difficulty breathing accompanied by a chest examination that indicates rales;
b. consolidation on chest x-ray; and
c. at least one indicator selected from the group consisting of: fever, high white blood cell count, and a productive cough.

20. The method according to claim 19, wherein the subject is symptomatic for influenza for more than 48 hours prior to the administration of the neuraminidase inhibitor and the antibiotic.

21. The method according to claim 20, wherein the neuraminidase inhibitor is selected from the group consisting of: oseltamivir phosphate, zanamivir and RJW-270201 (BCX-1812).

22. The method according to claim 21, wherein the nueraminidase inhibitor is oseltamivir phosphate which is administered orally and the at least one antibiotic is selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime,trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine.

23. A method of treating a secondary bacterial infection in a subject who acquires the bacterial infection as a sequelae to a viral influenza infection comprising administering a composition comprising a therapeutically effective amount of a neuraminidase inhibitor in combination with a therapeutically effective amount of at least one antibiotic to a subject who has been symptomatic for influenza for more than 48 hours prior to treatment.

24. The method according to claim 23, wherein the secondary bacterial infection is selected from the group consisting of: pneumonia, otitis media and sinusitis.

25. The method according to claim 24, wherein pneumonia is mediated by Streptococcus pneumoniae.

26. The method according to claim 24, wherein the neuraminidase inhibitor is selected from the group consisting of: oseltamivir phosphate, zanamivir and RJW-270201 (BCX-1812).

27. The method according to claim 26, wherein the nueraminidase inhibitor is oseltamivir phosphate and the at least one antibiotic is selected from the group consisting of: ceftriaxone, cefotaxime, vancomycin, meropenem, cefepime, ceftazidime, cefuroxime, nafcillin, oxacillin, ampicillin, ticarcillin, ticarcillin/clavulinic acid (Timentin), ampicillin/sulbactam (Unasyn), azithromycin, trimethoprim-sulfamethoxazole, clindamycin, ciprofloxacin, levofloxacin, synercid, amoxicillin, amoxicillin/clavulinic acid (Augmentin), cefuroxime,trimethoprim/sulfamethoxazole, azithromycin, clindamycin, dicloxacillin, ciprofloxacin, levofloxacin, cefixime, cefpodoxime, loracarbef, cefadroxil, cefabutin, cefdinir, and cephradine.
